(19) United States
(12) Patent Application Publication
(10) Pub. No.: US 2017/0362564 A1
(43) Pub. Date: Dec. 21, 2017

(54) SYSTEM AND PROCESS FOR LYSING A BIOLOGICAL SAMPLE

(71) Applicant: Commissariat A L'Energie Atomique et aux Energies Alternatives, Paris (FR)

(72) Inventors: Hadrien LAMOTTE, Villard Bonnot (FR); Jean-Luc ACHARD, Grenoble (FR); Jean-Maxime ROUX, La Tranche (FR)

(73) Assignee: Commissariat A L'Energie Atomique et aux Energies Alternatives, Paris (FR)

(21) Appl. No.: 15/627,474

(22) Filed: Jun. 20, 2017

(30) Foreign Application Priority Data
Jun. 20, 2016 (FR) 16 55703

(51) Int. Cl. C12N 1/06; C12N 13/00; C12M 1/00

(52) U.S. Cl. CPC C12N 1/066; C12M 47/06; C12N 13/00

(57) ABSTRACT

A process carried out for lysing biological species present in an aqueous solution and for recovering a biological material resulting from the lysis of the biological species, the process being carried out by using a system which includes a vessel delimiting a volume, a first electrode and a second electrode each including an end placed in the volume formed by the vessel, a voltage-pulse-generating circuit. The process includes applying at least one voltage pulse between the two electrodes with the voltage-pulse-generating circuit connected to the two electrodes, so as to bring about the creation of an electric arc between the end of the first electrode and the end of the second electrode and the generation of at least one shockwave in the solution.

### TECHNICAL FIELD OF THE INVENTION

The invention relates to a process for lysing biological species and for recovering the biological material resulting from the lysis and to a system for lysing a biological sample and for recovering the biological material resulting from the lysis of the biological species.

### PRIOR ART

The detection of pathogens in a biological sample is often carried out using heavy and relatively unsuitable equipment. The detection requires in particular a prior step of lysing the biological species containing the sample so as to release therefrom a biological material to be analysed.

There are several methods for lysing biological species. By way of example, they may be of:

chemical type, by adding reagents to the solution which contains the biological species to be lysed,

mechanical type, by milling, by laser-generated shockwaves or by ultrasound-generated shockwaves,

thermal type,

electrical type, by electroporation.

Patent application WO 2015/181743 A1 describes in particular a mechanism of mechanical lysis by milling. In said mechanism, the mechanical lysis is more particularly carried out by shearing between two walls, one of the two walls having a rough bearing surface.

Patent applications WO 2007/061943 A2 and US 2012/219987 A1 describe electroporation lysis solutions.

The various known solutions all have some advantages, but they also have a certain number of drawbacks listed below:

the chemical method requires the introduction of reagents into the solution, which has a tendency to pollute the solution.

the mechanical method by milling is difficult to carry out automatically.

the method by laser-generated shockwaves requires expensive equipment.

the method by ultrasound-generated shockwaves is difficult to carry out and generates noise.

the thermal method can lead to denaturation of the biological species.

the electrical method by electroporation, as described in the abovementioned prior documents, does not have a very satisfactory yield.

A first objective of the invention is thus to provide a process for lysing biological species contained in a solution and for recovering the biological material obtained at the end of the lysis, this process being reliable, easy to carry out and inexpensive, not requiring a high concentration of biological species and easily automated.

### SUMMARY OF THE INVENTION

This objective is achieved by means of a process used for lysing biological species present in an aqueous solution and for recovering a biological material resulting from the lysis of the biological species, said process being carried out by means of a system for lysing said biological species, said system comprising:

a vessel delimiting a volume,

a first electrode comprising an end placed in said volume formed by the vessel,

a second electrode comprising an end placed in said volume formed by the vessel and separated from the end of the first electrode by a non-zero separation distance,

the end of the first electrode and the end of the second electrode being configured and arranged so as to ensure between them a point effect in the presence of an electric field,

a voltage-pulse-generating circuit, said process comprising the following steps:

at least partially filling the volume of the vessel with the solution containing the biological species to by lysed such that the end of the first electrode and the end of the second electrode are immersed in said solution,

applying at least one voltage pulse between the two electrodes by means of the voltage-pulse-generating circuit connected to the two electrodes, said pulse being generated at a sufficient amplitude and for a sufficient period to bring about the creation of an electric arc between the end of the first electrode and the end of the second electrode, leading to the generation of at least one shockwave in said solution,

recovering the biological material resulting from the lysis of the biological species contained in the solution.

The solutions described in the abovementioned prior art do not allow the creation of an electric arc, the configuration of the electrodes not being suitable for this. In said documents, the electrodes are in fact arranged so as to present surfaces facing one another between which the electric field required for carrying out the electroporation is formed.

According to one particularity of the process, during the filling step, the vessel is partially filled with the solution containing the species to be lysed, in such a way as to leave a volume of air between the surface of the solution and the top of the vessel, thus allowing the creation of a gas bubble after the generation of said shockwave.

According to another particularity, during the filling step, the volume of the vessel is totally filled and it comprises a step of stopping the filling by capillary action.

According to another particularity, the step of recovering said biological material comprises a subsequent step of expulsion of the solution out of the vessel, caused by the generation of said shockwave.

According to one particular embodiment, the filling step is carried out according to the principle of communicating vessels.

Another objective is also to provide a novel system for lysing biological species contained in a solution, which makes it possible to overcome the prior art drawbacks. The system of the invention is in particular:

not very bulky,

easy to use since it is automatic,

of moderate cost,

not polluting for the solution,

of satisfactory yield,

of quite low energy cost,

not requiring a high concentration of biological species.

The invention also relates to a system for lysing a biological sample according to the process defined above, said system comprising:

a vessel delimiting a volume intended to be at least partially filled with a solution containing biological species to be lysed,

a first electrode comprising an end placed in said volume formed by the vessel,

a second electrode comprising an end placed in said volume formed by the vessel and separated from the end of the first electrode by a non-zero separation distance,

the end of the first electrode and the end of the second electrode being configured and arranged so as to ensure between them a point effect in the presence of an electric field,

a pulse-generating electric circuit connected to the first electrode and to the second electrode.

Preferentially, said largest dimension defining the transverse surface of each electrode in contact with the solution is for example less than 50% of said separation distance.

Advantageously, the end of the first electrode and the end of the second electrode are each in the form of a point.

Advantageously, each point is formed along an axis and the axis followed by the end of the first electrode and the axis followed by the end of the second electrode coincide.

Preferentially, the pulse-generating circuit comprises:

a high-voltage generator which has two power supply terminals each connected to a distinct electrode,

a capacitor connected, in parallel with the two electrodes, to the two terminals of the generator by means of a resistance,

a switch for controlling the expulsion of the capacitor and thus controlling the triggering of the voltage pulses between the two electrodes.

Advantageously, the high-voltage generator is arranged so as to provide a voltage of between 5 and 15 kV.

According to one particular embodiment, the system comprises at least one acoustic reflector, positioned so as to reflect each shockwave generated towards the interior of the volume of the vessel.

According to one particular embodiment, the vessel defines a volume which has a bottom and a neck defining an expulsion orifice.

Advantageously, the system comprises filling pipes connected to the vessel for feeding the vessel with solution containing biological species to be lysed according to the principle of communicating vessels.

Advantageously, said filling pipes are arranged so as to open out in the bottom of the vessel.

Advantageously, the expulsion orifice of the neck is proportioned to create a capillary valve stopping the filling of the vessel when the vessel is full up to the level of its neck.

### BRIEF DESCRIPTION OF THE FIGURES

Other characteristics and advantages will emerge in the detailed description which follows, given with regard to the attached drawings in which:

FIG. 1 represents, generally, the structure of the system of the invention, making it possible to carry out the process of the invention.

FIG. 2 represents a preferred embodiment of the pulse-generating circuit which is used.

FIG. 3 represents one particular embodiment of the system of the invention.

FIGS. 4A to 4C represent implementation variants of the system of the invention using reflectors or a particular arrangement of the vessel of the system of the invention.

FIG. 5 illustrates the various steps of the process of the invention, carried out for the lysis of the biological species by means of the system of the invention.

FIG. 6 represents an implementation variant of the system of the invention, enabling the lysis of the biological species contained in the solution and recovery of the lysed solution.

FIG. 7 represents a curve connecting the height of solution in the reservoir containing the reserve of solution to be lysed to the radius of the expulsion orifice present on the vessel.

FIG. 8 represents the various steps of the lysis and extraction process carried out by means of the system represented in FIG. 6.

FIG. 9 illustrates an example of implementation of the process of the invention.

### DETAILED DESCRIPTION OF AT LEAST ONE EMBODIMENT

The system of the invention is intended to allow lysis of the biological species contained in a sample in order to release therefrom a biological material to be studied. This biological sample is for example in the form of an aqueous solution which contains the biological species. The term “biological species” is intended to mean in particular microorganisms, cells, spores, bacteria, microalgae, fungi, viruses, etc. The term “biological material to be studied” is intended to mean for example nucleic acid (RNA, DNA) molecules derived from a cell, proteins, lipopolysaccharides (LPSs), lipoteichoic acids (LTAs), etc.

In the subsequent description, in a non-limiting manner, a biological sample comprising biological species present in a solution such as water will be taken as example.

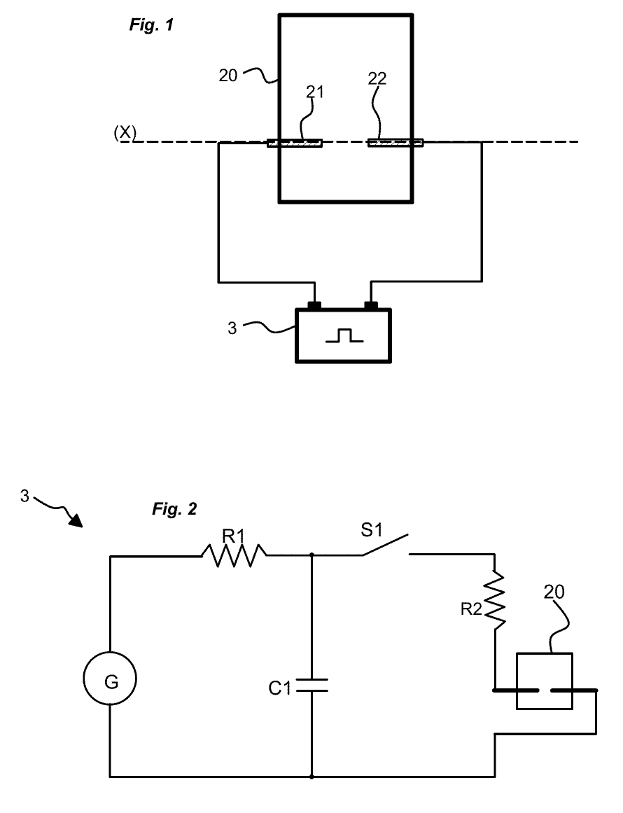

According to the invention, the lysis is carried out by electrical effect. With reference to FIG. 1, the system of the invention thus comprises:

a vessel 20 which defines a volume intended to receive the solution which contains the biological species to be lysed.

a first electrode 21 having an end placed in the volume of the vessel 20.

a second electrode 22 having an end placed in the volume of the vessel, the end of the second electrode 22 being positioned relative to the end of the first electrode at a determined non-zero separation distance e.

a voltage-pulse-generating electric circuit 3, connected to the first electrode 21 and to the second electrode 22.

According to the invention, it will be considered that the separation distance e mentioned in the present description is the shortest distance that separates the two ends of the electrodes 21, 22 immersed in the solution.

According to the invention, each electrode 21, 22 ends with at least one elongated part extending along a longitudinal axis.

Each electrode is configured and arranged so as to ensure, at its end, a point effect (known in the lightening conductor field) relative to the other when voltage pulses are applied between the two electrodes. Indeed, the point of these objects amplifies the electric field in their vicinity.

According to the invention, the end of the first electrode 21 which is in contact with the solution 4 to be lysed and the end of the second electrode 22 which is in contact with the solution to be lysed are each formed from a transverse surface, configured so as to ensure a point effect and to allow between them the generation of an electric arc.

The transverse surface of each electrode is advantageously configured in terms of dimensions and position relative to the transverse surface of the other electrode so as to ensure the generation of the electric arc between these two surfaces when voltage pulses of a determined level are applied.

The transverse surface of each electrode has a largest dimension which is small relative to the separation distance e separating the two ends of the electrodes. The separation distance e is preferentially between 1 and 4 mm. Said largest dimension of the transverse surface will preferentially be less than 50% of said separation distance e.

Advantageously, each electrode 21, 22 ends at its end with a point, forming said transverse surface for creating the electric arc. The two points are located facing one another. The two points are for example rectilinear in shape and each produced along said longitudinal axis. Advantageously, the axis followed by the point of the first electrode and the axis followed by the point of the second electrode coincide (see axis (X) on the figures). Upstream of its point, each electrode may have any shape.

Moreover, it should be noted that the electric arc created between the ends of the two electrodes 21, 22 will be perfectly located in the solution and will always and only be generated in one and the same zone located between the two points and not randomly inside the tank 20 containing the solution. This architecture thus allows reproducibility in the application of the lysis. The electric arc generation zone is defined by a delimited volume in the solution to be lysed (necessarily less than the total volume of the vessel), around the rectilinear segment joining the ends of the two electrodes 21, 22. The electric arc created begins at the end of one of the two electrodes and ends at the end of the other electrode.

On the basis of this system, the invention consists in creating one or more shockwaves in the aqueous solution 4 which contains the biological samples, for the purpose of weakening and/or destroying walls of the biological species present in the solution 4 and thus releasing the biological material.

For this, the pulse-generating electric circuit 3 generates voltage pulses within the solution 4 containing the biological species to be lysed, resulting in the flashover of the solution, that is to say in the appearance of an electric arc between the ends of the two electrodes 21, 22. During the appearance of this electric arc, a first series of shockwaves comprising at least one shockwave is generated in the solution 4, making it possible to lyse the biological species and thus to release the biological material.

According to the configuration, there then follows the appearance of a gas bubble (mainly water vapour) in the zone of the solution where the electric arc is formed. This gas bubble grows to its maximum size and then rapidly collapses, therefore generating a second series of shockwaves. Moreover, under certain conditions and for some biological species to be lysed, it would also be possible to observe the creation of a cavitation cloud in the solution above the electrodes, made up of several cavitation bubbles generated by pressure reductions associated with each series of shockwaves. Each of the bubbles of the cavitation cloud then collapses, thereby locally generating multiple shockwaves.

Of course, it is understood that, in order to obtain the expected effect, various electrical parameters will have to be adjusted in a manner adapted to the operating conditions. This will in fact involve applying at least one voltage pulse of amplitude and duration sufficient to bring about the creation of the electric arc between the ends of the two electrodes 21, 22 immersed in the solution. These parameters of the voltage pulse include, in addition:

the separation distance e between the two ends of the electrodes 21, 22, said distance having to allow the creation of the electric arc when a voltage pulse is generated.

the conductivity of the solution 4 in which the biological species are placed, said conductivity having an influence on the generation of the electric arc between the two electrodes 21, 22 and will thus necessarily have an influence on the separation distance e between the two ends of the electrodes 21, 22.

Moreover, for the purpose of optimizing the system of the invention, this will also involve attending to the duration of the ascending front of the pulse and the duration of the descending front of the pulse. In a non-limiting manner, the ascending front will have a duration of less than 1 microsecond, preferentially less than 500 nanoseconds.

Advantageously, in order to allow the creation of the gas bubble after the first series of shockwaves, it is necessary to take into account the following parameters:

The height hsol of solution above the electrodes, said height having to be adjusted so as to allow sufficient growth of the gas bubble in the solution after the formation of the vapour channel. Said height will have to be adjusted as a function of the biological species to be lysed. For example, if the biological species are microalgae, the height hsol of solution above the electrodes must be sufficient to allow the development of a cavitation cloud (i.e. the development of microbubbles following the passing of the first series of shockwaves) above the electrodes. If the biological species to be lysed are vegetative bacteria, the height hsol of solution must be sufficient to allow the development of the gas bubble between the electrodes, but must remain relatively low in order to make it possible to confine the second series of shockwaves created by the collapse of the gas bubble.

The height hair of air above the surface of the solution, said height influencing the size of the gas bubble generated.

The cross section of opening of the volume of the vessel on its upper part, for example defined by its length L and its width I if the cross section of opening is rectangular, or its diameter if the cross section is circular.

On the basis of these various parameters, by way of example and in a non-limiting manner, a lysis is carried out under the conditions described above.

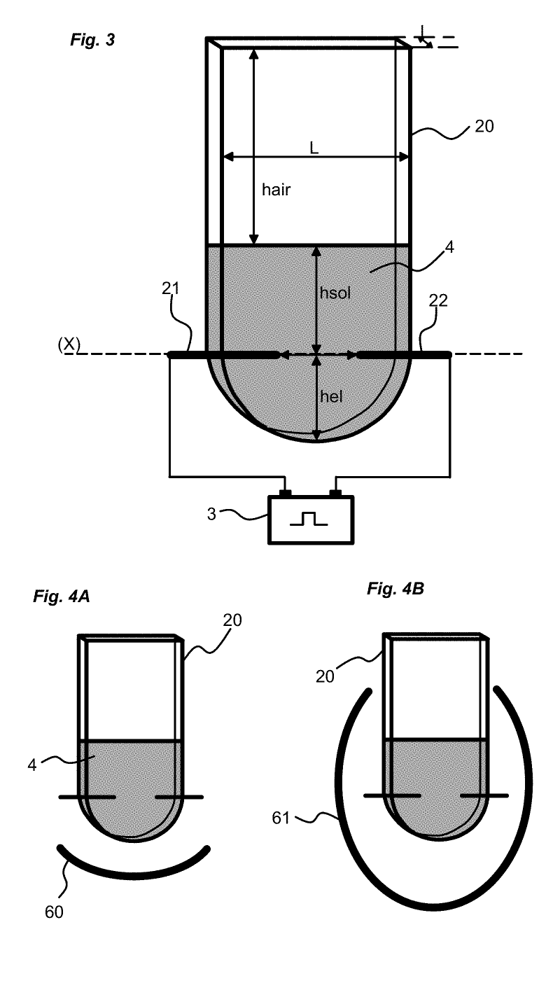

With reference to FIG. 3, the vessel 20 has, for example, a longitudinal cross section defining a U-shaped volume and a cross section of opening in the transverse plane which is rectangular in shape. The vessel 20 will for example be produced by any known conventional technique, such as for example machining, moulding, printing, etc. It will for example be obtained by machining in a plastic (for example of Topas (registered trademark) 5013 type) or by stereolithography printing in a polymer (for example of Perfactory (registered trademark) Envision LS600 type). The vessel 20 will preferentially be sealed closed at its top, by means of a suitable cover.

As described above, the two electrodes 21, 22 each end for example with a straight point at their end. The two points thus formed are for example placed facing one another in the volume of the vessel, along one and the same axis (X) and apart from one another by a suitable separation distance e.

With reference to FIG. 2, the pulse-generating circuit 3 comprises for example:

A high-voltage generator G which has two power supply terminals each connected to a distinct electrode 21, 22.

A capacitor C1 connected, in parallel with the two electrodes, to the two terminals of the generator G by means of a resistance R1.

A switch S1 for controlling the discharge of the capacitor C1 and thus controlling the triggering of the voltage pulses between the two electrodes 21, 22.

A protective resistance R2 placed in series with the electrodes 21, 22 for preventing any risk of short-circuit of the capacitor C1.

In addition, according to the biological species to be lysed and the intended application, with reference to FIG. 3, the operational conditions defined in one of the two tables presented below will be adhered to.

For the analysis/identification (for example of bacteria, fungi, viruses or m icroalgae):

| Parameter | Definition | Values |
|---|---|---|
| hₛₒₗ | Height of solution above | Between 4 mm and |
| | the electrodes | 10 mm - preferably 7 mm |
| hₐᵢᵣ | Height of air present | Between 2 mm and |
| | above the surface of the | 10 mm - preferably 5 mm |
| | solution | |
| hₑₗ | Height of the electrodes | Between 3 mm and |
| | relative to the bottom of | 6 mm - preferably 3 mm |
| | the volume of the vessel | |
| L | Length of the cross | Between 6 mm and |
| | section of opening of the | 10 mm - preferably 6 mm |
| | vessel | |
| I | Width of the cross | Between 1 mm and |
| | section of opening of the | 4 mm - preferably 2 mm |
| | vessel | |
| e | Separation distance | Between 1 mm and |
| | between the points of | 4 mm - preferably 2 mm |
| | the electrodes | |

For the extraction of compounds of interest (for example the content of microalgae), in order to observe the generation of a cavitation cloud above the electrodes, it will be necessary to adhere to the following operating conditions:

| Parameter | Definition | Values |
|---|---|---|
| hₛₒₗ | Height of solution above | Between 10 mm and |
| | the electrodes | 100 mm - preferably 30 mm |
| hₐᵢᵣ | Height of air present | Between 2 mm and |
| | above the surface of the | 10 mm - preferably 5 mm |
| | solution | |
| hₑₗ | Height of the electrodes | Between 3 mm and |
| | relative to the bottom of | 10 mm - preferably 3 mm |
| | the volume of the vessel | |
| L | Length of the cross | Between 6 mm and |
| | section of opening of the | 20 mm - preferably 6 mm |
| | vessel | |
| I | Width of the cross | Between 1 mm and |
| | section of opening of the | 5 mm - preferably 2 mm |
| | vessel | |
| e | Separation distance | Between 1 mm and |
| | between the points of | 5 mm - preferably 2 mm |
| | the electrodes | |

In order to generate the shockwaves under such operating conditions, the voltage generator G is for example capable of supplying a voltage of several kilovolts, for example between 5 and 15 kV, preferentially approximately 10 kV, in the form of pulses each having a duration of a few tens of microseconds each, between 20 and 40 μs, preferentially approximately 30 μs. These operating parameters of the voltage generator G must however be adjusted to the conductivity of the solution containing the biological species that are present in the vessel 20. They will in particular be sufficient to allow the flashover of a solution 4 having a conductivity that is not very high, typically between 30 and 500 S.m⁻¹. During the flashover of the solution, the generator delivers a current peak having a very high intensity that can range up to 10⁴ A.

With reference to FIG. 5, under such conditions, the principle of lysis implemented in the process of the invention is the following:

The solution 4 which contains biological species 40 to be lysed is placed in the vessel 20 of the system of the invention (step E0).

The pulse-generating circuit (not represented in FIG. 5) generates a high-voltage pulse in accordance with the characteristics defined above, generating electric arcs 50 between the two electrodes (step E1).

The electric arcs generated create a conducting channel 51 between the two electrodes, leading to the flashover of the solution (step E2).

The flashover of the solution leads to the generation of at least a first series of shockwaves 52 (step E2) leading to a first lysis by enabling weakening and/or destruction of the walls of the biological species 40 present in the solution 4.

At the end of the shockwave, a vapour bubble 53 is created between the two electrodes (step E3), promoted by the free space present in the vessel above the surface of the solution. Under certain conditions, the first series of shockwaves will be able to lead to the generation of cavitation bubbles above the electrodes (step E3′).

The bubble of vapour 53 generated gradually grows up to a maximum size (step E4).

After having reached its maximum size, the bubble of vapour 53 implodes (step E5). Likewise, each cavitation bubble generated above the electrodes implodes (not represented).

During the implosion of the bubble of vapour 53 and the cavitation bubbles, a second series of shockwaves 54 is generated, leading to a second lysis of the biological species 40 present in the solution 4 (step E6).

New cavitation bubbles may appear at the end of the second series of shockwaves. By imploding, they will lead to the creation of new local shockwaves.

The automatable nature of the lysis principle described above enables the process of the invention to readily allow recovery of the biological material resulting from the lysis of the biological species.

The principle here is to use the generation of the shockwaves to expel at least one part of the volume of the solution present in the vessel 20 and thus to easily recover a solution which contains lysed biological species without any human intervention.

Moreover, in order to optimize the implementation of this extraction process, two principles described below can be taken in combination.

The first principle is that of communicating vessels. Indeed, it is envisaged to fill the vessel 20 with the solution which contains the biological species to be lysed by using the principle of communicating vessels. The system of the invention thus comprises at least one filling pipe which opens into the lower part of the vessel 20, preferentially at the bottom thereof, and which is fed by a reservoir of solution, containing the biological species to be lysed and placed above the top of the vessel 20, at a height suitable for bringing about filling of the vessel by the principle of communicating vessels.

The second principle employed uses capillary action. It involves using a capillary valve to naturally stop the filling of the vessel 20 when the latter is full. For this, the vessel 20 comprises, in its upper part, a neck defining a solution expulsion orifice. This orifice has a diameter suitable for stopping the filling of the vessel with the solution which contains the biological species to be lysed when the volume of the vessel is full and when the surface area of the solution reaches the narrowest cross section of the neck.

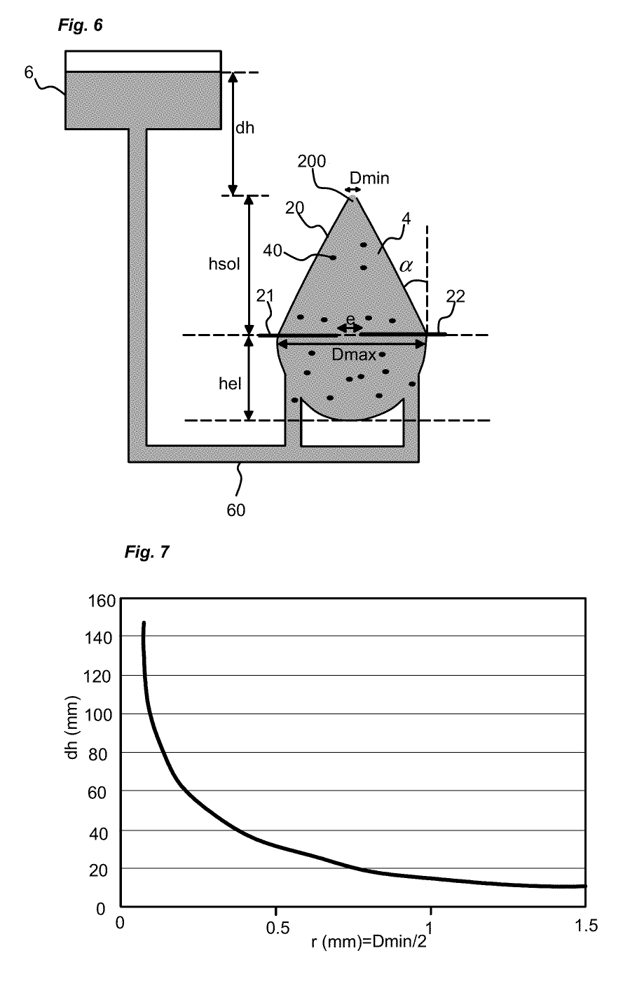

FIG. 6 thus represents the improved system for lysing the biological species contained in the solution and recovering the lysed solution, this system arranged in particular for implementing the two principles described above.

In this FIG. 6, the vessel 20 is for example in the form of a carboy comprising a lower part of which the bottom is rounded and an upper part which is conical in shape and the top of which is pierced by an axial orifice 200. This axial orifice 200 is proportioned so as to allow the implementation of the capillary valve principle described above.

As described above, the system comprises for example a filling pipe 60 fed by a reservoir 6 and opening at two distinct points in the lower part of the vessel 20, preferentially under the electrodes and thus in the bottom of the vessel.

As described above, the reservoir 6 is filled with a solution comprising biological species to be lysed. In order to implement the principle of communicating vessels, this reservoir must necessarily be placed above the top of the vessel in order to be able to completely fill it.

Moreover, in order to implement the capillary valve principle, it will be necessary to ensure a sufficient pressure of liquid at the level of the expulsion orifice 200. FIG. 7 thus shows a curve indicating a relationship between the difference in height (dh), between the surface of solution in the reservoir 6 and the top of the vessel 20, and the radius (r) of the expulsion orifice 200 present on the vessel 20 (also defined by the diameter Dm in in FIG. 6).

The electrodes 21, 22 are placed as previously described and the voltage-pulse-generating circuit 3 remains identical.

With reference to FIG. 6, in order to carry out the process reliably and efficiently, the system used will preferentially be proportioned in the following way:

| Parameter | Definition | Values |
|---|---|---|
| hₛₒₗ | Height of solution above | Between 1 mm and |
| | the electrodes | 8 mm - preferably 3 mm |
| hₑₗ | Height of the electrodes | Between 3 mm and |
| | relative to the bottom of | 10 mm - preferably 4 mm |
| | the volume of the vessel | |
| Dmax | Maximum diameter of | Between 5 and 10 mm - |
| | the vessel at the level of | preferably 6 mm |
| | the base of its cone | |
| Dmin | Diameter of the | Between 0.2 mm and |
| | expulsion orifice | 1.2 mm - preferably |
| | | between 0.5 and 0.8 mm |
| α | Angle defining the | Between 20 and 50° - |
| | conical part of the | preferably 30° |
| | carboy | |
| e | Separation distance | Between 1 mm and |
| | between the points of | 4 mm - preferably 2 mm |
| | the electrodes | |

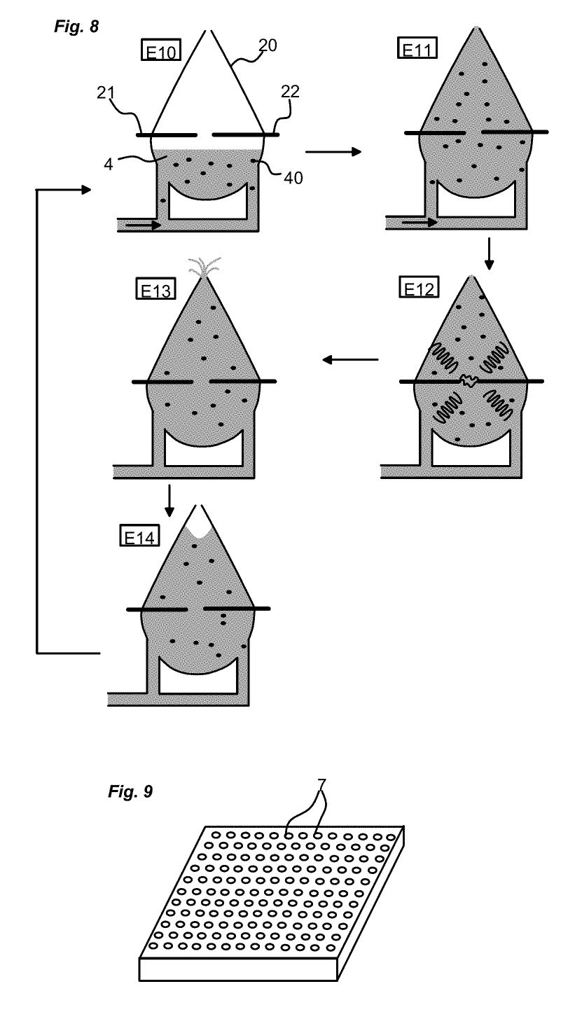

FIG. 8 illustrates the various steps carried out using the system described above and represented in FIG. 6.

By the principle of communicating vessels, the vessel 20 is filled from the reservoir with solution containing biological species to be lysed—step E10.

The capillary valve at the top of the vessel stops the filling of the vessel when the vessel is full—step E11.

As described above, the steps of lysing the biological species contained in the solution are then carried out. This thus involves generating a first series of shockwaves by flashover of the solution by applying voltage pulses. This principle is described above in connection with FIG. 5—step E12.

The generation of the first series of shockwaves causes the capillary valve to burst open and thus a part of the lysed solution to be expelled out of the vessel. This sample of lysed solution can thus be easily recovered, without having to handle the vessel. This step occurs simultaneously with the preceding step of generating the first series of shockwaves—step E13.

Once the expulsion is finished, the filling of the vessel 20 can start again as in step E10.

The process described above is in particular efficient for lysing biological species such as microalgae. These microalgae will for example be of Nannoch/oropsis or Phaeodactylum type, from which lipids or chlorophyll are extracted.

The frequency of the pulses emitted by the voltage-pulse-generating circuit 3 is adjusted to the rate of filling of the vessel. In order to expel a maximum of lysed solution out of the vessel 20, it is necessary to wait for the vessel to be completely filled. A solution level detector in the vessel may be provided in order to be sure that the vessel 20 is indeed full when the voltage-pulse-generating circuit 3 is activated. A programmable automated device will be perfectly suitable for carrying out such an operating sequence.

Moreover, as represented in FIG. 9, in order to maximize the cost-effectiveness of the process, it would be possible to install, in parallel, several lysis and recovery units 7, each in the form of a system as described above in connection with FIG. 6, for example with a reservoir 6 common to all units. Each unit will be able to produce a determined volume of lysed solution. By using a system having the characteristics described above, each unit will for example be able to produce a volume of 5 microlitres per second. By placing a hundred or so units in parallel, it is thus possible to obtain a lysed solution volume of 16 litres per hour, operating at a frequency of two high-voltage pulses per second.

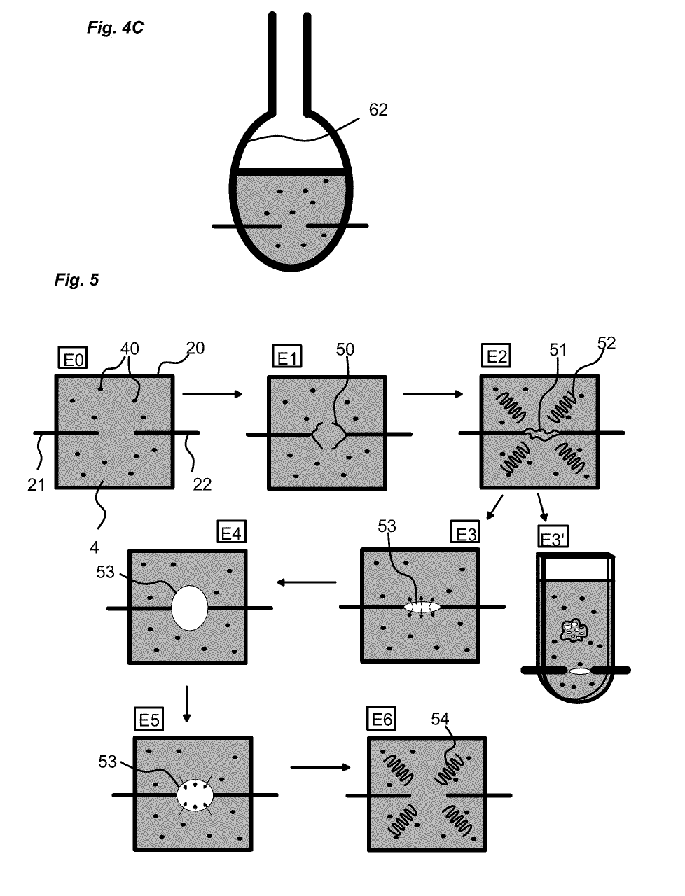

According to the invention, with reference to FIGS. 4A to 4C, in order to improve the efficiency of the system of the invention and to take better advantage of each shockwave generated, it is possible to implement a reflection of the shockwaves. For this, it involves for example adding one or more acoustic reflectors of suitable shape around the volume defined by the vessel 20 in order to focus the shockwaves inside the vessel. FIGS. 4A to 4C thus present some distinct embodiments.

In FIG. 4A, a partial reflector 60 in the shape of an arc of a circle, placed under the bottom of the vessel, is used.

In FIG. 4B, an oval-shaped reflector 61 encompasses the entire volume defined by the vessel.

In FIG. 4C, the internal volume defined by the vessel is in the shape of an ellipse itself forming an acoustic reflector 62 for the shockwaves generated.

The solution of the invention described above thus has numerous advantages, in particular:

It is simple to use since it does not require complex or expensive equipment.

It can be used automatically since it involves simply controlling the pulse-generating circuit.

It causes no pollution of the solution.

It makes it possible to easily recover lysed solution without having to handle the vessel.

Even treated in small amount, the pulse generation frequency makes it possible to lyse and to recover a large amount of solution, thus making it exploitable on a larger scale.

1. A process carried out for lysing biological species present in an aqueous solution and for recovering a biological material resulting from the lysis of biological species, said process being carried out with a system for lysing said biological species, said system comprising:
a vessel delimiting a volume,
a first electrode comprising an end placed in said volume formed by the vessel,
a second electrode comprising an end placed in said volume formed by the vessel and separated from the end of the first electrode by a non-zero separation distance,
the end of the first electrode and the end of the second electrode being configured and arranged so as to ensure between them a point effect in the presence of an electric field,
a voltage-pulse-generating circuit,
wherein said process comprises the following steps:
at least partially filling the volume of the vessel with the solution containing the biological species to be lysed such that the end of the first electrode and the end of the second electrode are immersed in said solution,
applying at least one voltage pulse between the two electrode with the voltage-pulse-generating circuit connected to the two electrodes, said pulse being generated at a sufficient amplitude and for a sufficient period to bring about the creation of an electric arc between the end of the first electrode and the end of the second electrode, leading to the generation of at least one shockwave in said solution,
recovering the biological material resulting from the lysis of the biological species contained in the solution.

2. The process according to claim 1, wherein, during the filling step, the vessel is partially filled with the solution containing the species to be lysed, in such a way as to leave a volume of air between the surface of the solution and the top of the vessel, thus allowing the creation of a gas bubble after the generation of said shockwave.

3. The process according to claim 1, wherein, during the filling step, the volume of the vessel is completely filled and wherein said process further comprises a step of stopping the filling by capillary action.

4. The process according to claim 3, wherein the step of recovering said biological material comprises a subsequent step of expulsion of the solution out of the vessel, caused by the generation of said shockwave.

5. The process according to claim 3, wherein the filling step is carried out by the principle of communicating vessels.

6. A system for lysing a biological sample said system comprising:
a vessel delimiting a volume intended to be at least partially filled with a solution containing biological species to be lysed,
a first electrode comprising an end placed in said volume formed by the vessel,
a second electrode comprising an end placed in said volume formed by the vessel and separated from the end of the first electrode by a non-zero separation distance (e),
the end of the first electrode and the end of the second electrode being configured and arranged so as to ensure between them a point effect in the presence of an electric field,
a pulse-generating electric circuit connected to the first electrode and to the second electrode.

7. The system according to claim 6, wherein a largest dimension defining the transverse surface area of each electrode in contact with the solution is less than 50% of said separation distance.

8. The system according to claim 6, wherein the end of the first electrode and the end of the second electrode are each in the form of a point.

9. The system according to claim 8, wherein each point is formed along an axis and wherein the axis followed by the end of the first electrode and the axis followed by the end of the second electrode coincide.

10. The system according to claim 6, wherein the pulse-generating circuit comprises:
a high-voltage generator which has two power supply terminals each connected to a distinct electrode,
a capacitor connected, in parallel with the two electrodes, to the two terminals of the generator by means of a resistance,
a switch for controlling the discharge of the capacitor and thus controlling the triggering of the voltage pulses between the two electrodes.

11. The system according to claim 10, wherein the high-voltage generator is arranged so as to supply a voltage of between 5 and 15 kV.

12. The system according to claim 6, comprising at least one acoustic reflector positioned so as to reflect each shockwave generated towards the interior of the volume of the vessel.

13. The system according to claim 6, wherein the vessel defines a volume which has a bottom and a neck defining an expulsion orifice.

14. The system according to claim 13, comprises filling pipes connected to the vessel for feeding the vessel with solution containing biological species to be lysed according to the principle of communicating vessels.

15. The system according to claim 14, wherein said filling pipes are arranged so as to open out in the bottom of the vessel.

16. The system according to claim 13, wherein the expulsion orifice of the neck is proportioned to create a capillary valve stopping the filling of the vessel when the vessel is filled to the level of its neck.
